(19) United States
(12) Patent Application Publication
(10) Pub. No.: US 2022/0315511 A1
(43) Pub. Date: Oct. 6, 2022

(54) HARVESTER DECARBOXYLATOR WITH TERPENE EXTRACTION SYSTEM

(71) Applicant: AG QUIP, LLC, St. Petersburg, FL (US)

(72) Inventor: Daniel T. Querrey, Pittsford, VT (US)

(21) Appl. No.: 17/708,399

(22) Filed: Mar. 30, 2022

Related U.S. Application Data
(60) Provisional application No. 63/168,254, filed on Mar. 30, 2021.

(51) Int. Cl. C07C 29/147; B01D 17/02; B01D 5/00

(52) U.S. Cl. CPC C07C 29/147; B01D 17/0217; B01D 17/0214; B01D 5/0057

(57) ABSTRACT

An arrangement of a mobile harvester decarboxylator with a terpene collector includes a heated enclosure for use in the field with terpene collectors coupled to ducted fume hoods of the heated enclosure. The terpene collector may include a chilled coil that condenses and separates oil-based terpenes from water-based terpenes via a centrifugal effect and/or immiscibility.

### CROSS RELATED APPLICATIONS

This application claims priority to U.S. provisional application No. 63/168,254, which was filed on Mar. 30, 2021 and is hereby incorporated by reference in its entirety herein.

### FIELD OF THE INVENTION

The field relates to extraction of aromatic compounds from plants, especially the extraction of terpenes.

### BACKGROUND

Essential oils are produced by glandular trichomes and other secretory structures, specialized secretory tissues mainly diffused onto the surface of plant organs, particularly flowers and leaves, thus exerting a pivotal ecological role in plant.

Terpenes are known to be extracted from plants. It is thought, without being limiting in any way, that many terpenes are lost when harvesting and drying plants, however. Expeller pressing of oils from plants and solvent extraction are usually not able to be performed in the field where organic plants originate. Therefore, plants are often hung to dry to prevent fungus and bacteria from ruining the crop before extraction of oils from oil producing plants. This results in the loss of a substantial portion of certain terpenes that otherwise might be extracted and isolated.

Terpenes are aromatic compounds found in many plants and are necessarily volatile and difficult to extract in commercial production. These aromatic compounds create characteristic scents of many plants, such as Cannabis, pine, mint, hops, roses, orange peel, and lavender. The scent of many essential oils is related to these terpenes, and these terpenes have many uses if they can be extracted without damaging the terpenes or terpenoids. Due to volatility, applicant has determined, 25% of oil-based terpenes of plants are lost when such plants are hang dried, and 80% of water-soluble terpenes are lost if plants are hang dried (i.e. air dried).

In addition, essential oils have been used, since ancient times, in many different traditional healing systems all over the world, because of their biological activities. Many preclinical studies have documented antimicrobial, antioxidant, anti-inflammatory and anticancer activities of essential oils in a number of cell and animal models, also elucidating their mechanism of action and pharmacological targets.

Often, a process of decarboxylation is required to convert plant acids into useful essential oils and the like. Decarboxylation is a chemical reaction that removes a carboxyl group and releases carbon dioxide (CO2). Usually, decarboxylation refers to a reaction of carboxylic acids, removing a carbon atom from a carbon chain. Decarboxylators are known that decarb acids to form oils such as essential oils. In processing CBD, a cannabidiolic acid (CBDA) is converted to cannabidiol by a process of decarboxylation.

### SUMMARY

A new process and machine make extraction of terpenes in the field or adjacent to a field commercially viable. Harvesting to extraction occurs within minutes or a few hours rather than days. This process dramatically improves the quality and quantity of terpenes extracted from plant biomass, such as flowers and leaves. The handling and management of plants is improved from harvesting through extraction, which provides surprising and unexpected yields of terpenes in the field. Other known processes fail to collect such terpenes when plants are dried prior to extraction. Laboratory scale processes are not analogous to field extraction, which handles large volumes of biomass to extract much smaller quantities of terpenes. In one example, one or more of the extracts are decarboxylated as part of the process, while still in the field location. The processes described in this application have been shown to greatly increase the amount of terpenes collected during harvesting in the field.

For example, a harvesting and extraction process reduces the time between harvesting and extraction using a mobile harvester decarboxylator that may include a terpene extraction system. In the first step, plants that are to be processed are harvested from a field. The plants may be transported to a debucker. The debucking may be capable of removing some or all of the stems from the plants, if the stems don't contain a sufficient amount of the desired terpenes. By debucking, the efficiency of removal of terpenes from the remaining portions of the plants may be increased as the mass of the biomass containing terpenes is reduced by removing flowers and/or leaves from stems. The plants may be transported to the debucker manually, by a harvester or by separate vehicles, such as harvesting trucks, in which the harvester/harvester debucker unload plant biomass. A debucker may output the portion of the plants to be further processed to another vehicle or to a conveyor that transports the plant biomass to be further processed. A processing station receives the plant biomass. In this way, the stems may be debucked from the biomass to be processed. Herein, debucking, debucked and debuck are verbs for removing some or all of the stems from other plant material before processing the other plant material.

Plants may be directed to one of a plurality of processing steps in the field. In one example, portions of the plants may be further separated for processing, such as removing flowers for processing separately from other plant biomass. In an alternative example, the flowers and/or other plant biomass is separated sufficiently for terpene extraction following a debucking process, various examples of debucking are known in the industry. In an alternative process, no debucking is necessary and the entire plant is directed for processing, as is, from the harvester. This is not known in the industry. In one example, a harvester may function as a rough debucker by selectively removing portions of plants from which terpenes will be extracted, for example, as part of a process of collecting those portions of plants from a field, without a separate debucking apparatus/process.

The desired plant biomass for extraction may be directed into a heated enclosure, as quickly as possible, by this extraction process being performed in the field. Some terpenes that might have been desirably extracted may be lost after harvesting or debucking or disturbing the plants in any way before extraction can be performed, if extraction is performed in a process distant from the field where harvesting of plants is performed. These volatile, desirable terpenes may be captured by the examples provided herein, because the apparatus and process reduce delays in extraction between harvest and extraction and reduce the time between debucking and extraction, if debucking is an intermediate step in the process.

Following extraction of terpenes in a heated enclosure, the plant biomass may be directed to a chute or airlock that removes the plant biomass from the heated enclosure while reducing the loss of any volatilized terpenes within the heated enclosure. The plant biomass may be directed by a conveyor to a bulk bagger, which bags the biomass for extraction of less volatile oils or the like at a later time. Alternatively, the plant biomass may be directed to an expeller or solvent extractor for removal of less volatile oils or the like without an intermediate bagging or transport step. The process of decarboxylating may occur during the step of terpene extraction, for example.

In one example, the more volatile terpenes are released by the plant biomass during heating and processing within the heated enclosure. For example, the processing may comprise tumble drying in a drum or tumble drying by a conveyor turning process. In one example, the conveyor turning process has a substantial advantage by turning and drying the plant biomass without causing undesirable damage to the plants. For example, tumble drying caused damage to plants that resulted in the production of small fragments of plant biomass, which fouled the heated enclosure and became overheated by resting on heated surfaces or the like. Avoiding dust and possible combustion of such plant dust that could cause hazardous combustion is an important consideration in choosing extraction methods. The heated enclosure is referred to herein as a decarboxylator, because it may be heated to a temperature that is known to decarboxylate certain plant acids. The decarboxylator includes a terpene extraction and recovery system that permits terpenes to be collected from the biomass that would otherwise be lost during drying of the biomass and/or decarboxylating of plant acids.

The volatile terpenes released by the plant biomass within the heated enclosure may be directed to one or more ducted hoods. In one example, a plurality of ducted hoods directs the volatilized terpenes to one or more chillers. For example, each ducted hood may be coupled to its own, dedicated chiller, or one or more of the ducted hoods may direct volatilized terpenes to a common chiller. The chiller or chillers cool and separate the terpenes into oil-based terpenes and water-based and/or water-soluble terpenes. The cooler and drier air may be fed back into the heated enclosure. For example, the cooler, dried air may be preheated prior to reintroducing it back into the heated enclosure.

The entry point into the heated enclosure may be at the top portion of one end of an elongated enclosure and may use a mechanism to prevent heated air and vapor in the enclosure from freely escaping at the point of entry. For example, a spring-loaded or gravity closing flap or the like may block the entry point into the enclosure. Alternatively, an airlock, such as a rotary drum airlock may be used for both the entry point and the exit point from the heated enclosure. In one example, the airlock comprises vanes within a drum that rotate around a center portion from which the vanes extend radially within the drum. As the vanes rotate, plant biomass may be deposited between pairs of vanes through an opening in the drum. As the vanes rotate about a central axis of the drum, the volume between the vanes is closed by the solid sidewalls of the drum and the plant biomass rotates with the vanes until the plant biomass exits from an opening in the bottom of the drum. For example, the opening in the bottom of the drum may empty the plant biomass onto a conveyor that directs the plant biomass to the next step in the process.

In one example, the next step in the process is extraction of oils that are less volatile than the terpenes extracted during heating in the heated enclosure. For example, these oils may form during decarboxylating of plant acids and may be extracted by an expeller press, a steam assisted expeller press and/or solvent extraction method, with or without heat/pressure. An expeller press may put the plant biomass under pressure to separate oils from the plant biomass, for example. In one example, an expeller press includes a screw type expeller. In one example, steam is injected into the plant biomass during expeller pressing of the plant biomass, which extracts additional oil not extracted by pressure alone. In yet another example, a solvent is added to the plant biomass to extract or assist in extracting the oil from the plant biomass. For example, alcohol may be used as a solvent. Following extraction, the alcohol may be removed from the oil by vacuum and/or heating of the oil. A chiller may be used to extract any remaining terpenes or oils from the alcohol. In one example, the alcohol is reused as a solvent after processing the alcohol to recover any terpenes or other oils from the air/alcohol. For example, the chiller may be a cryo-chiller. The alcohol may be separated from air and water in an alcohol recovery process before being reused.

For example, an extraction and decarboxylation process comprises: disposing a decarboxylator in a location adjacent to a field; harvesting plant biomass from the field; introducing plant biomass harvested from the field in the step of harvesting into the decarboxylator, without delays due to air drying or transport of the plant biomass to a remote location, distant from the field, wherein the plant biomass comprises at least one terpene that is volatile at ambient temperature and pressure that would be lost by delaying due to air drying or transport of the plant biomass to a remote location, distant from the field, and at least one plant acid that is capable of being decarboxylated; heating the plant biomass in a heated enclosure at an elevated temperature for decarboxylation in a range from 107 to 118 degrees centigrade, wherein the at least one plant acid is decarboxylated by the elevated temperature in the heated enclosure, and the at least one terpene is volatilized; holding the plant biomass within the heated enclosure at the elevated temperature for a duration, wherein decarboxylation and volatilization are substantially complete; circulating the air within the heated enclosure; and separating the at least one terpene from water vapor and air within the decarboxylator by circulating the air through a terpene condenser, capturing the at least one terpene, whereby the quality and quantity of the at least one terpene is improved compared to any process using extended air drying plant biomass prior to capturing of terpenes or any decarboxylating processes that are performed remote from the location adjacent to the field being harvested. The step of circulating may redirect air separated from the at least one terpene during the step of separating and may feed the separated air back into the heated enclosure. A step of debucking may occur during the step of harvesting, or substantially no debucking may occur prior to the step of holding, unlike other known processes that require thorough debucking prior to decarboxylation. A step of turning of the plant biomass may occur during the step of holding. The process may further comprise using an entry airlock and an exit airlock to limit the loss of heat and volatile terpenes from the air inside the heated enclosure. A step of extraction of at least one decarboxylated plant acid may occur after the at least one plant acid is substantially decarboxylated. The process may further comprise bulk bagging of the plant biomass prior to the step of extraction of the decarboxylated at least one plant acid. Alternatively, the step of extraction of the at least one decarboxylated plant acid may comprise expelling oils, solvent extraction or a combination of expelling oils and solvent extraction without any step of bulk bagging prior to the step of extraction. The step of extraction of the at least one decarboxylated plant acid may comprise steam-assisted expelling of oils. The at least one terpene may include a water-based or water-soluble terpene and an oil-based terpene, and the step of separating may separate the oil-based terpene from the water-based or water-soluble terpene at the field location. In one example, a mobile apparatus is used in a field location for decarboxylating at least one plant acid contained in plant biomass harvested from the field and capturing at least one volatile terpene from the plant biomass harvested from the field, the apparatus may comprise: a heated enclosure with an entry into the heated enclosure for the plant biomass, and exit from the heated enclosure for the plant biomass, and a means for turning or tumbling the plant biomass during heating of the plant biomass, wherein the heated enclosure is elongated along the direction of a length of the heated enclosure; a heating unit for heating the enclosure to a temperature in a range to decarboxylate the at least one plant acid; and a terpene separator that captures the at least one volatile terpene and separates the at least one volatile terpene from the air at the field location, wherein the terpene separator comprises a chiller condenser. The means for turning or tumbling the plant biomass may comprise a plurality of conveyors, wherein one of the plurality of conveyors transports the plant biomass along the length of the heated enclosure before depositing the plant biomass on another of the plurality of conveyors, wherein the plant biomass is turned, exposing a new surface of the plant biomass on the another of the plurality of conveyors, and the another of the plurality of conveyors extends beyond the length such that the another of the plurality of conveyors catches the plant biomass from the one of the plurality of conveyors and redirects the plant biomass back through the length of the heated enclosure, extending the duration that the plant biomass is held within the heated enclosure, for example. Alternatively, a drum tumbler may be used for tumbling plant biomass. The terpene separator may be arranged such that oil-based terpenes are separated from water-based and water-soluble terpenes, for example. The apparatus may further comprise one or more ducted hoods, and at least one of the one or more ducted hoods may be in fluid communication with the terpene separator. Alternatively, each of a plurality of the one or more ducted hoods may be in fluid communication with the terpene separator, and the terpene separator may be in common to each of the plurality of one or more hoods. The entry into the heated enclosure may be disposed at the top portion of one end of the heated enclosure, and the entry may comprise a spring-loaded or gravity closing flap that reduces the escape of heated air and vapors from the heated enclosure. Alternatively, the entry into the heated enclosure may comprise a rotary drum airlock having a plurality of vanes rotating within a drum such that plant biomass is deposited between pairs of the plurality of vanes through an opening in the drum, and as the plurality of vanes rotate about a central axis of the drum, the volume between the vanes is closed by the solid sidewalls of the drum, rotating the plant biomass through the drum to an exit opening in a bottom portion of the drum such that the plant biomass enters the heated enclosure while leakage of heat and vapor in the heated enclosure is limited by the rotary drum airlock. An expeller press may be arranged such that the plant biomass is pressed under pressure to separate oils from the plant biomass after plant acids are substantially decarboxylated, for example. The expeller press may comprise steam injection into the plant biomass during expeller pressing of the plant biomass, extracting additional oil from the plant biomass that would not be extracted by pressure alone. Air in the heated enclosure may be held at a temperature in the range of 107 to 118 degrees centigrade, for example.

### BRIEF DESCRIPTION OF THE DRAWINGS

The following drawings are illustrative examples and do not further limit any claims that may eventually issue.

FIG. 1 illustrates a schematic example of a mobile decarboxylator arranged in a mobile harvester decarboxylator system.

FIG. 2 illustrates an example of a process flow diagram for a mobile decarboxylator.

FIG. 3 illustrates a partial side view of an example of a mobile decarboxylator.

FIG. 4 illustrates a continuation of the partial side view of the example in FIG. 3.

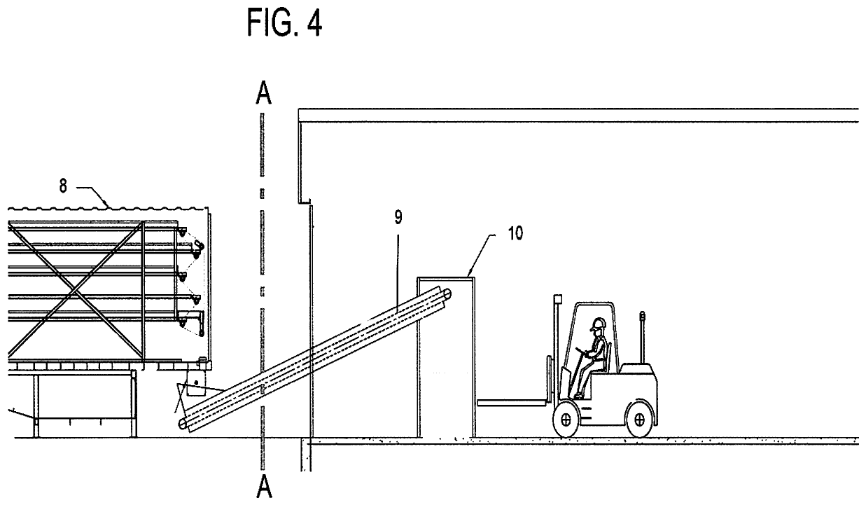

FIG. 5 illustrates a schematic example of a decarboxylator/extractor.

FIG. 6 illustrates a side view of a conveyor system.

FIG. 7 illustrates an end view of FIG. 6.

FIG. 8 illustrates another example of a decarboxylator/extractor.

FIG. 9 illustrates an example of a terpene collector.

When the same reference characters are used, these labels refer to similar parts in the examples illustrated in the drawings.

### DETAILED DESCRIPTION

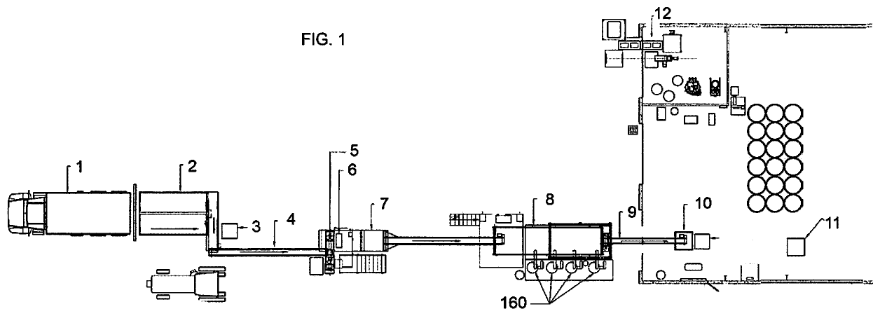

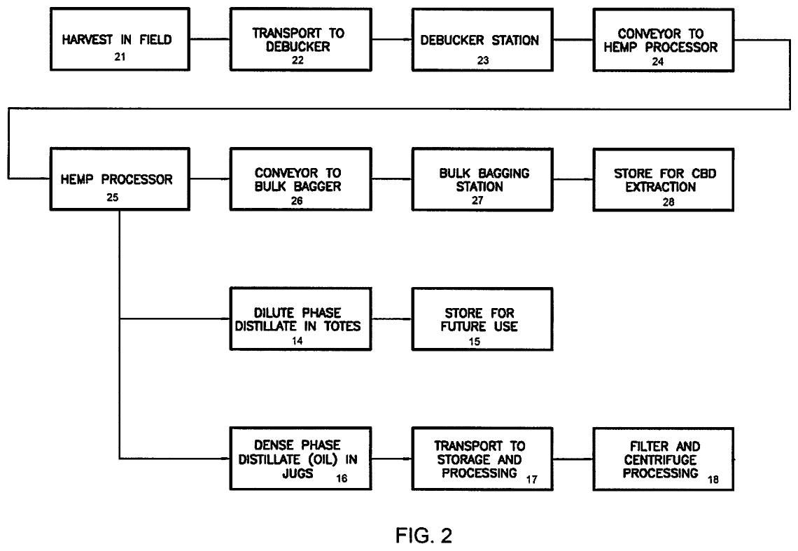

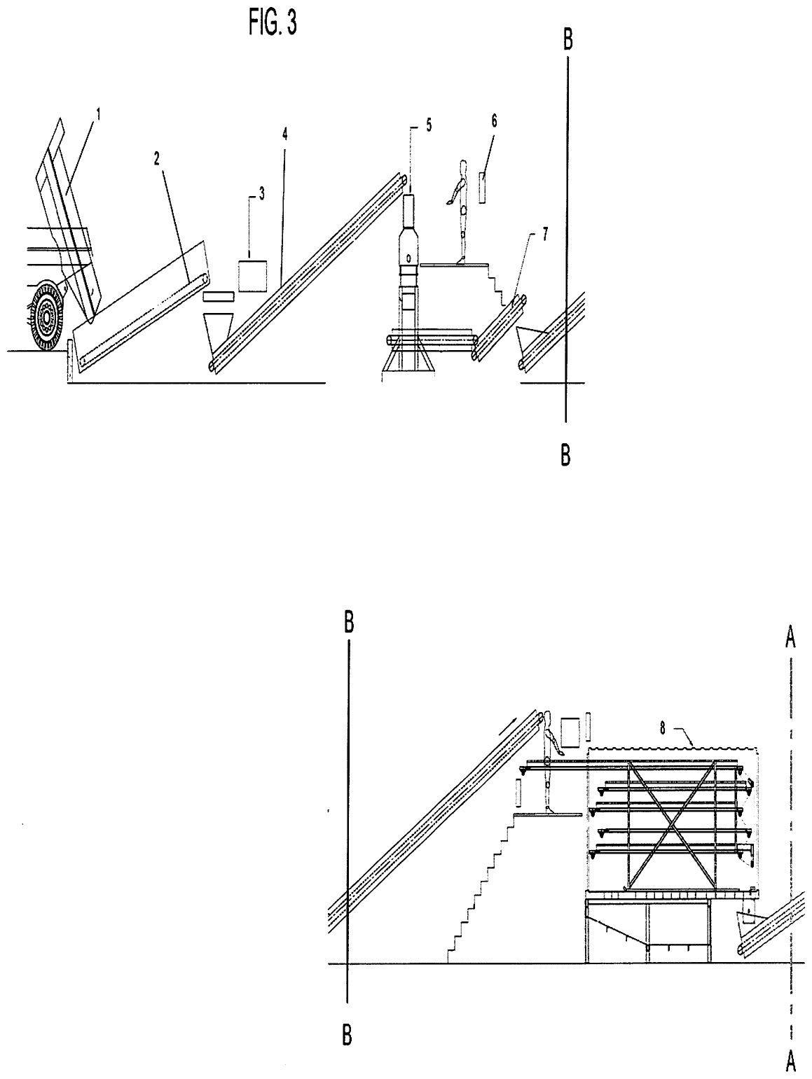

In one example, as illustrated in FIGS. 1 and 3, a mobile harvester decarboxylator arrangement 10 is shown for use in the field according to a method, such as illustrated in the flow chart of FIG. 2. The arrangement includes a transport vehicle 1, such as a dump truck, emptying a load of harvested plant biomass on a transfer conveyor 2. The plant biomass may be inspected at a first inspection station 3 before being transferred to an inclined conveyor 4, which delivers the biomass to a free water press 5. The biomass is deposited onto a second inclined conveyor 7, which transports the biomass in a heated enclosure 8, which may decarboxylate and extract terpenes from the biomass. The mobile arrangement quickly and efficiently translates the biomass through the preparatory steps to the heated enclosure 8 in minutes, rather than hours or days that it takes to transport plant biomass to centralized processing facilities. The unexpected result is a 5-fold increase in usable water-based terpenes and a 25% increase in oil-based terpenes compared to conventional drying, transport and centralized processing of plants.

The harvester decarboxylator system comprises a heated enclosure 8 through which the biomass passes while terpenes and water are evaporated from the plants, such as the flowers and leaves of the plants. For example, terpenes may be extracted from Cannabis, pine, mint, hops, roses, orange peel, and lavender. This list is not all inclusive. The terpenes for many essential oils may be extracted using the system.

In one example, a 72 cubic foot heated enclosure 8 may extract 1.5 liters per hour of terpenes with about 10% being oil-based terpenes and 90% being water-based terpenes (with or without some residual oil mixed with the water). In one example, the water-based terpenes are soluble in the water. There may be less than 1% by weight of terpenes in a water-terpene solution or water-based terpene extract, for example.

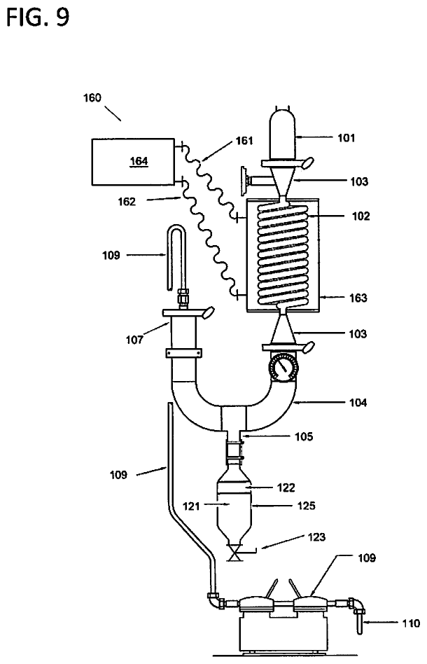

For example, the terpene collection system of the heated enclosure may comprise a hood 56 and a duct vented by a duct blower 55 and/or vacuum pump 109. For example, a vacuum pump may be an oil free pump, such as a dairy pump, to prevent contamination of the air/terpenes. The duct extends from the heated enclosure and connects the enclosure to a terpene collector. A detailed example of a terpene collector is provided in FIG. 9, for example.

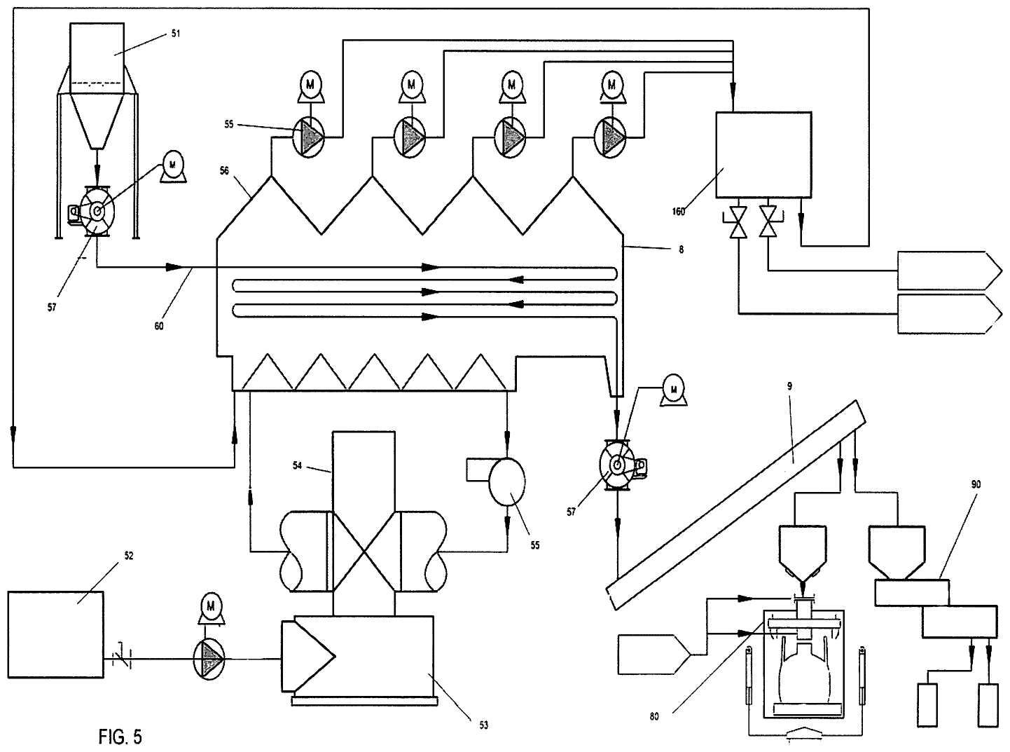

In one example of the process, as illustrated in the flow diagram of FIG. 2, plant biomass is harvested 21 in the field using a mobile harvester decarboxylator. After harvesting with a harvester, the biomass may be transported 22 to a free water removal/debucking operation 23 for optionally removing some or all of the stems and removing free water. The remaining biomass is disposed on an inclined conveyor for conveying 24 the biomass to the heated enclosure for decarboxylation and terpene extraction 25, after which the biomass is conveyed 26 to a bulk bagging station for bagging 27 for later extraction of additional oil using an expeller, steam expeller and/or solvent expeller. Alternatively, the biomass may be fed directly to an expeller, steam expeller and/or solvent expeller as schematically shown in FIG. 5. Bagger stations are known in the art. In one example, the expeller is a steam expeller 90 that injects steam into a jacketed screw while passing biomass through the screw expelling oil and emitting a biomass cake. The method includes a step of separating the water-based terpenes 14 from the oil-based terpenes 16. The water-based terpenes may be stored 15 for later processing or use. The oil-based terpenes may be transported 17 for further processing, such as filtering and centrifuge processing of the oil 18, separating the oil-based terpenes into separate products prior to analytical testing 19 to determine type, amount and quality of the terpenes collected. For example, the decarboxylator comprises a method of terpene extraction that maximizes the quantity and/or quality of the terpenes extracted from plant biomass.

For example, the heated enclosure may be heated in a temperature range from 225 to 245 degrees Fahrenheit (107 to 118 Centigrade), which may heat the plant materials to a temperature of about 138 degrees F. (50 degrees Centigrade). Keeping the plants at about this temperature at about one atmosphere within the enclosure is preferred. In one example, a vacuum pump, such as a commercial, oil free dairy pump, or a duct blower is used to circulate air from the hood/duct through a terpene collection chiller and back to the enclosure. In one example, a 72 cubic foot enclosure comprises a plurality of hoods/ducts, such as four hoods/ducts, each of the hoods/ducts directing air through a respective terpene collection chiller. The vacuum pressure of each pump or blower should be sufficient to turn over the air inside the container in a range between 3 times per hour to 12 times per hour, more preferably 5 times per hour to 10 times per hour, even more preferably 6 times per hour to 10 times per hour. The optimal range of the turnover rate may be observed. If steam leaks from the enclosure, the pressure in the container is too great and the vacuum pressure should be increased. Excessive outside air leaking into the enclosure requires additional heating to keep the plant matter within the correct temperature range within 20 degrees Fahrenheit of 138 degrees F., more preferably within 10 degrees F. of 138 degrees F. Excessive introduction of outside air means that the vacuum pressure should be reduced.

In one example, each of the terpene collection chillers comprise an inlet, which may comprise a 3 inch sanitary pipe is coupled to a ¾ inch tubular metal coil by a 3 inch to ¾ inch concentric reducer. For example, the metal may be a stainless steel, such as 304 stainless. The coil may have an inner diameter of 6 inches and a 22 foot coil length, in one example, which produces a centrifugal effect within the optimal operating parameters of the vacuum pump/duct that draws volatile volatile vapors and air through the coil. In this example, the coil length of the tube may be determined as the inner diameter of the coil times 30. For example, the dryer enclosure volume in cubic feet may be used to determine the coil inner diameter by dividing the enclosure volume in cubic feet by 100. The chiller coil may pass through a volume of liquid chilled to a temperature by a chiller to a temperature below the dew point. For example, a temperature 30 degrees Fahrenheit below the dew point may be chosen to extract both oil-based and water-based terpenes. In one example, the coil is coupled to a pressure differential collection chamber by a ¾ inch to 3 inch expander (which may be the same as the reducer). For example, a terpene collector may be coupled to the pressure differential chamber for draining oil and water-based terpenes from the pressure differential chamber to the terpene collector. The terpene collector may be a transparent material such as glass and may one or more bleed valves for bleeding off water-based terpenes, oil-based terpenes or both thereof. In one example, the oil-based terpenes are immiscible in water and separate from the water. For example, passing through the coil may exert a centrifugal force that helps to separate the oil from the water, which remain separated as gravity directs the oil and water into the terpene collector. The oil floats on the water. A valve or stop cock on the bottom of the collector may be used to drain water-based terpenes, for example.

In one example, a half inch tube, such as a steel tube, preferably stainless steel, extends upwardly coupling an upwardly extending portion of the pressure differential chamber to the vacuum pump/duct blower, which recirculates the now dryer air back into the heated enclosure. The air may be preheated by heaters prior to introducing the air back into the enclosure, for example.

For example, the pressure differential chamber may be a U-shaped tube having a diameter 4 times the diameter of the tubular metal coil that passes through the chiller. Any similar volume differential will produce the same pressure differential in the pressure differential chamber, regardless of shape. The drain in the pressure differential chamber couples the pressure differential chamber to the terpene collector.

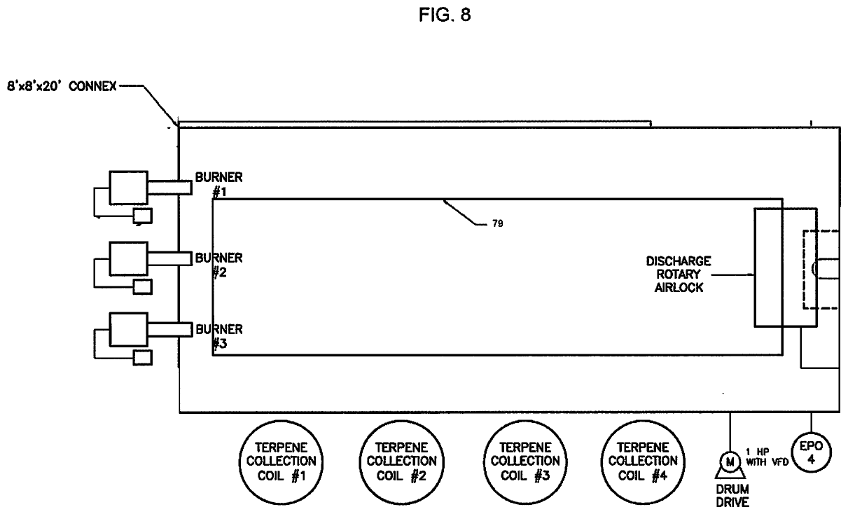

In the example of FIG. 8, the heated enclosure comprised a rotary drying drum mounted within the enclosure and coupled to a motor for rotating the rotary drying drum. The rotary drying drum is coupled to a motor by a chain or other temperature-insensitive coupling that rotates the rotary drying drum within the heated enclosure, constantly turning the plant matter while the plant matter transits the rotary drying drum. In one example, the rotary drying drum may comprise vanes, a sloped conical surface, or the like, which moves the plant matter through the drum from an inlet chute to an exit airlock. In the example of FIG. 8, the heated enclosure comprises a rotary drum 79 instead of a conveyor system for turning the biomass. The vanes or the like direct the plant matter from the chute to the airlock while the rotation of the rotary drum turns the plant matter making it easier for warm, dryer air to reach all of the biomass evaporating the volatile terpenes and water and drying the biomass of the plant matter, such as stems, leaves, flowers and the like. While the rotary drum turned the biomass providing a viable method of extracting terpenes and water from the biomass of plant matter, a shortcoming of the rotary drum method was excessive damage to sensitive portions of the plants, such as flowers or the like, that resulted in collection of pollen, dust and small fragments of the plant escaping from the drum. Some of these smaller fragments collect on the surfaces, such as the floor of the enclosure, and could become heated to a temperature that might scorch or even burn these smaller fragments. This may require periodic cleaning to avoid dangerous combustion of these smaller fragments, if they were to dry out. While the drying temperature should remain below the combustion temperature, the creation of dust that is potentially combustible is a hazard that must be avoided. Dust explosions in grain elevators and the like is a known hazard during process of plant matter.

In the example of FIG. 5, a transfer bin 51 transfers the biomass to an airlock 57, which deposits the biomass on the first conveyor 60. A plurality of ducted hoods 56 with duct blowers 55 direct fumes from the enclosure to a terpene collector 160, where terpenes are separated as water based and oil based. The biomass exits the enclosure through another airlock 57. The enclosure is heated using fuel stored in fuel storage 52 that is combusted in a burner 53 that heat recirculating air passing through a heat exchanger 54 by way of a circulating fan 55. The biomass that exits the enclosure is deposited on a conveyor 9 and alternatively is fed to a bagging station 80 or is directly fed to an extraction hopper for oil extraction using a steam expeller 90, for example.

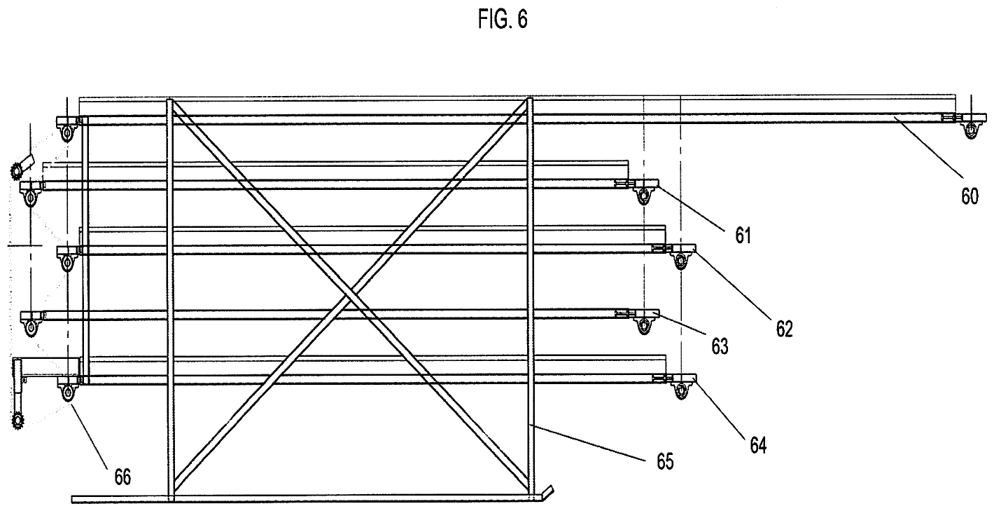

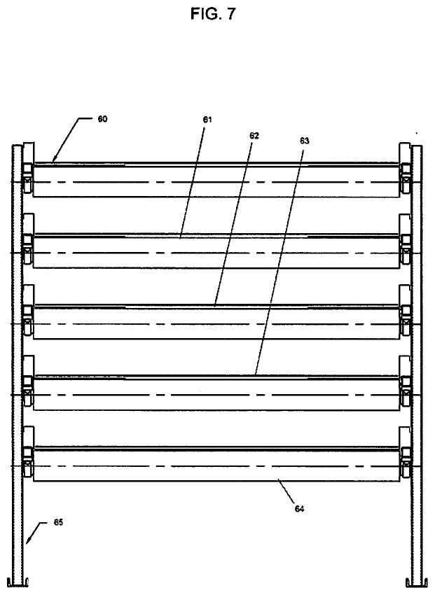

The inlet chute or airlock of the heated enclosure in the examples of FIGS. 3, 5, 6 and 7, the plant biomass is introduced into the heated enclosure and onto the first conveyor 60. As illustrated in the example of FIGS. 6 and 7, the conveyor system has a plurality of conveyors 60, 61, 62, 63, 64 coupled by a frame 65 to the heated enclosure and coupled together by a drive mechanism 66. The frame 65 may be fixed within the heated enclosure 8. A first conveyor 60 may extend to where an airlock deposits the biomass, conveying the biomass into the heated enclosure and depositing the biomass on a second conveyor 61 that extends beyond the end of the first conveyor 60. This turns the biomass for better drying. The second conveyor conveys the biomass in the opposite direction of the first conveyor due to a chain driven conveyor driving system 66. The third conveyor 62 extends beyond the second conveyor, turning the biomass and redirecting the biomass in the opposite direction. Likewise the fourth conveyor 63 and fifth conveyor 64 turn and redirect the biomass back through the elongated enclosure for decarboxylating, drying and extracting terpenes from the biomass as it traverses the enclosure back and forth from one conveyor to the next, until the last conveyor, which may deposit the biomass in an airlock for exiting the heated enclosure, decarboxylator, terpene extractor system.

In the example of FIG. 9, a terpene collector 160 comprises a chiller 16 operatively coupled 161, 162 by chiller lines to a coil enclosure 163 to chill a stainless steel coil having 13 six inch diameter coils. Fumes, water vapor and air enter the coil through tube 101 and reducer 103 and exit through expander 103 to a collection system 104, 105, 125, which comprises a U-shaped tube coupled to a dairy vacuum pump 109 by vacuum line 109. The vacuum line 109 couples to the upwardly extending end of the U-shaped tube 107. Terpenes are discharged through drain 105 to a glass collecting container 125. The immiscible oils 122 are separated from the condensing water-based terpenes 121 by centrifugal forces in the coil 102 and do not re-emulsify. One or more drain cocks 123 may be used to drain the water-based terpenes, oil-based terpenes or both. A recirculation line 110 redirects now dryer air back to the heated enclosure, closing the loop.

This detailed description provides examples including features and elements of the claims for the purpose of enabling a person having ordinary skill in the art to make and use the inventions recited in the claims. However, these examples are not intended to limit the scope of the claims, directly. Instead, the examples provide features and elements of the claims that, having been disclosed in these descriptions, claims and drawings, may be altered and combined in ways that are known in the art.

What is claimed is:

1. An extraction and decarboxylation process comprises:
disposing a decarboxylator in a location adjacent to a field;
harvesting plant biomass from the field;
introducing plant biomass harvested from the field in the step of harvesting into the decarboxylator, without delays due to air drying or transport of the plant biomass to a remote location, distant from the field, wherein the plant biomass comprises at least one terpene that is volatile at ambient temperature and pressure that would be lost by delaying due to air drying or transport of the plant biomass to a remote location, distant from the field, and at least one plant acid that is capable of being decarboxylated;
heating the plant biomass in a heated enclosure at an elevated temperature for decarboxylation in a range from 107 to 118 degrees centigrade, wherein the at least one plant acid is decarboxylated by the elevated temperature in the heated enclosure, and the at least one terpene is volatilized;
holding the plant biomass within the heated enclosure at the elevated temperature for a duration, wherein decarboxylation and volatilization are substantially complete;
circulating the air within the heated enclosure; and
separating the at least one terpene from water vapor and air within the decarboxylator by circulating the air through a terpene condenser, capturing the at least one terpene, whereby the quality and quantity of the at least one terpene is improved compared to any process using extended air drying of plant biomass prior to capturing of terpenes or any decarboxylating processes that are performed remote from the location adjacent to the field being harvested.

2. The process of claim 1, wherein the step of circulating redirects air separated from the at least one terpene during the step of separating and feeds the separated air back into the heated enclosure.

3. The process of claim 1, wherein a step of debucking occurs during the step of harvesting.

4. The process of claim 1, wherein substantially no debucking occurs prior to the step of holding.

5. The process of claim 1, wherein a step of turning of the plant biomass occurs during the step of holding.

6. The process of claim 1, further comprising using an entry airlock and an exit airlock to limit the loss of heat and volatile terpenes from the air inside the heated enclosure.

7. The process of claim 1, further comprising a step of extraction of at least one decarboxylated plant acid after the at least one plant acid is substantially decarboxylated.

8. The process of claim 7, further comprising bulk bagging of the plant biomass prior to the step of extraction of the decarboxylated at least one plant acid.

9. The process of claim 7, wherein the step of extraction of the at least one decarboxylated plant acid comprises expelling oils, solvent extraction or a combination of expelling oils and solvent extraction without any step of bulk bagging prior to the step of extraction.

10. The process of claim 7, wherein the step of extraction of the at least one decarboxylated plant acid comprises steam-assisted expelling of oils.

11. The process of claim 1, wherein the at least one terpene includes a water-based or water-soluble terpene and an oil-based terpene, and the step of separating separates the oil-based terpene from the water-based or water-soluble terpene at the field location.

12. A mobile apparatus for use in a field location for decarboxylating at least one plant acid contained in plant biomass harvested from the field and capturing at least one volatile terpene from the plant biomass harvested from the field, the apparatus comprising:
a heated enclosure comprising an entry into the heated enclosure for the plant biomass, and exit from the heated enclosure for the plant biomass, and a means for turning or tumbling the plant biomass during heating of the plant biomass, wherein the heated enclosure is elongated along the direction of a length of the heated enclosure;
a heating unit for heating the enclosure to a temperature in a range to decarboxylate the at least one plant acid; and
a terpene separator that captures the at least one volatile terpene and separates the at least one volatile terpene from the air at the field location, wherein the terpene separator comprises a chiller condenser.

13. The apparatus of claim 12, wherein the means for turning or tumbling the plant biomass comprises a plurality of conveyors, wherein one of the plurality of conveyors transports the plant biomass along the length of the heated enclosure before depositing the plant biomass on another of the plurality of conveyors, wherein the plant biomass is turned, exposing a new surface of the plant biomass on the another of the plurality of conveyors, and the another of the plurality of conveyors extends beyond the length such that the another of the plurality of conveyors catches the plant biomass from the one of the plurality of conveyors and redirects the plant biomass back through the length of the heated enclosure, extending the duration that the plant biomass is held within the heated enclosure.

14. The apparatus of claim 12, wherein the terpene separator is arranged such that oil-based terpenes are separated from water-based and water-soluble terpenes.

15. The apparatus of claim 14, further comprising one or more ducted hoods, and at least one of the one or more ducted hoods are in fluid communication with the terpene separator.

16. The apparatus of claim 14, wherein each of the one or more ducted hoods is in fluid communication with the terpene separator, and the terpene separator is common to each of the one or more terpene separators.

17. The apparatus of claim 12, wherein the entry into the heated enclosure is disposed at the top portion of one end of the heated enclosure, and the entry comprises a spring-loaded or gravity closing flap that reduces the escape of heated air and vapors from the heated enclosure.

18. The apparatus of claim 12, wherein the entry into the heated enclosure comprises a rotary drum airlock having a plurality of vanes rotating within a drum such that plant biomass is deposited between pairs of the plurality of vanes through an opening in the drum, and as the plurality of vanes rotate about a central axis of the drum, the volume between the vanes is closed by the solid sidewalls of the drum, rotating the plant biomass through the drum to an exit opening in a bottom portion of the drum such that the plant biomass enters the heated enclosure while leakage of heat and vapor in the heated enclosure is limited by the rotary drum airlock.

19. The apparatus of claim 12 further comprising an expeller press arranged such that the plant biomass is pressed under pressure to separate oils from the plant biomass after plant acids are substantially decarboxylated.

20. The apparatus of claim 19, wherein the expeller press comprises steam injection into the plant biomass during expeller pressing of the plant biomass, extracting additional oil from the plant biomass that would not be extracted by pressure alone.

21. The apparatus of claim 12, wherein air in the heated enclosure is held at a temperature in the range of 107 to 118 degrees centigrade.
